(19) United States
(12) Patent Application Publication
(10) Pub. No.: US 2004/0260309 A1
(43) Pub. Date: Dec. 23, 2004

(54) Introduction apparatus

(76) Inventor: Brian M. Packard, Monticello, MN (US)

Correspondence Address: Sean D. Solberg, Esq., DORSEY & WHITNEY LLP, Intellectual Property Department, 50 South Sixth Street, Suite 1500, Minneapolis, MN, 55402-1498, US

(21) Appl. No.: 10/818,089

(22) Filed: Apr. 5, 2004

Related U.S. Application Data
(60) Provisional application No. 60/460,696, filed on Apr. 4, 2003.

(51) Int. Cl. A61M 25/00

(52) U.S. Cl. 606/108

(57) ABSTRACT

The present invention is an apparatus for introducing a medical device into a catheter. The invention includes an introduction apparatus having an elongated tubular member, a first connection element, and a second connection element.

### CROSS-REFERENCE TO RELATED APPLICATION(S)

This application claims priority from U.S. provisional application No. 60/460,696, filed Apr. 4, 2003, which is hereby incorporated by reference in its entirety.

### FIELD OF THE INVENTION

The present invention relates to an apparatus for introducing a medical device into a catheter. It further relates to an apparatus for introducing medical devices that are flexible or have geometries that don't facilitate introduction.

### BACKGROUND OF THE INVENTION

Catheters and other similar medical devices are typically introduced into vessels by pushing the distal end of the device forward using the catheter shaft as support. The medical devices that are introduced in this manner may include intravascular devices, expanded nitinol meshes, balloon catheters, or similar devices that require exterior support to allow for advance of the device. Further, the devices include those where loading an element from the distal end is preferred as not to damage the primary catheter tip or intravascular portion.

Often the introduction and advancement of such a device into a vessel is difficult because the device is either too flexible or has a geometry that doesn't facilitate introduction. Further, loading such a device may damage the primary tip or, in the case of an intravascular device, the intravascular portion.

There is a need in the art for an apparatus that allows for the introduction and advancement of catheters and other medical devices into vessels. There is a further need for an apparatus that allows for the protection of the primary tip or any distal portions of the device.

### BRIEF DESCRIPTION OF THE DRAWINGS

FIG. 1 is a side view of an introduction apparatus, according to one embodiment of the present invention.

FIG. 2A is a cutaway side view of a first connection element, according to one embodiment of the present invention.

FIG. 2B is a cutaway side view of a first connection element connected to a catheter, according to one embodiment of the present invention.

FIG. 3A is a cutaway side view of a first connection element, according to an alternative embodiment of the present invention.

FIG. 3B is a cutaway side view of a first connection element connected to a catheter, according to an alternative embodiment of the present invention.

FIG. 4 is a flow chart of a method of using an introduction apparatus, according to one embodiment of the present invention.

FIG. 5 is a side view of an introduction apparatus having a tubular arm, according to one embodiment of the present invention.

FIG. 6 is a flow chart of a method of using an introduction apparatus, according to one embodiment of the present invention.

FIG. 7A is a perspective view of a portion of a tubular member, according to one embodiment of the present invention.

FIG. 7B is a flow chart of a grooving process, according to one embodiment of the present invention.

FIG. 8 is a flow chart of a method of adding external elements to a tubular member, according to one embodiment of the present invention.

### DETAILED DESCRIPTION

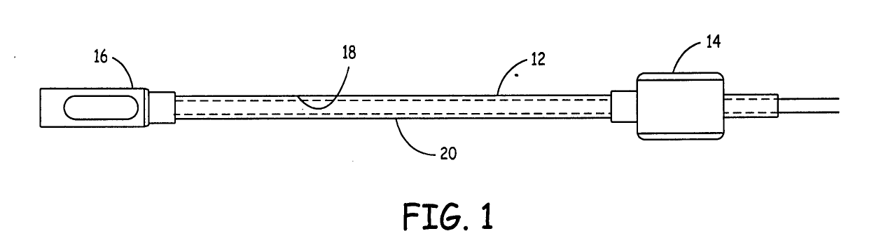

FIG. 1 depicts an introduction apparatus 10 according to one embodiment of the invention. The introduction apparatus has an elongated tubular member 12 and a first connection element 14 and a second connection element 16.

The first connection element 14, according to one embodiment, is associated with the elongated tubular member 12 at a distal portion of the tubular member 12. According to one embodiment, the first connection element 14 is located just proximal to the distal end of the tubular member. The first connection element 14 is configured to be attachable to a catheter. According to one embodiment, the first connection element is a lure lock. Alternatively, the first connection element is a snap or press fit. In a further alternative, the first connection element 14 can be any known connection device.

The location of the first connection element 14 just proximal to the distal end of the tubular member 12 allows the elongated tube to extend into the catheter and provide a relatively seamless introduction of a medical device into the catheter. In a further embodiment, the first connection element 14 is located anywhere in the distal portion of the tubular member 12. Alternatively, the first connection element 14 is at the distal end of the tubular member.

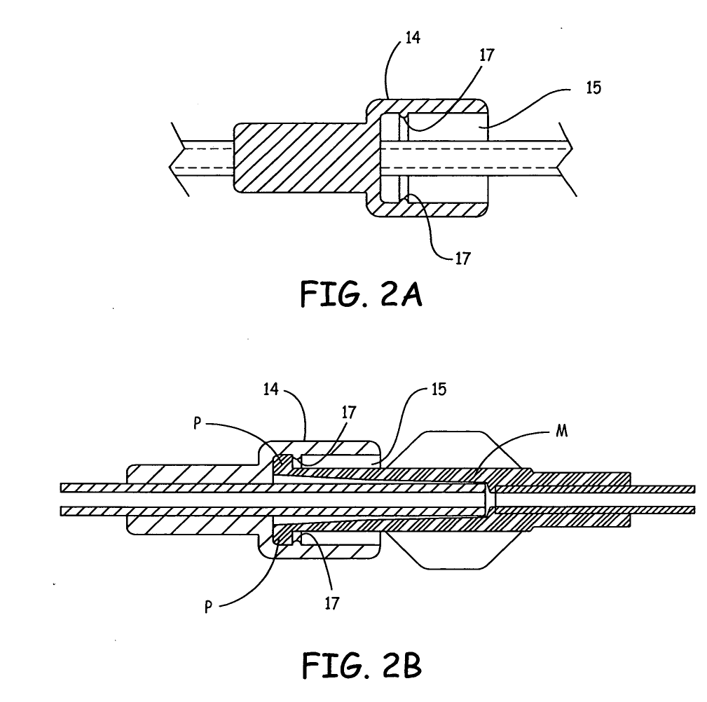

FIG. 2A depicts a cutaway side view of a first connection element 14, according to one embodiment of the present invention. FIG. 2B depicts a cutaway side view of the first connection element 14 of FIG. 2A in mated connection with a male portion M of a catheter, according to one embodiment of the present invention. The connection element 14, according to one embodiment, is a “female” connection element having an opening 15 at one end configured to receive an appropriate “male” connection portion of a catheter. Inside the opening 15, the connection element 14 has two small protruding elements 17. The protruding elements 17 are configured to contact protruding elements P on the male element M such that the male element M and the first connection element 14 are held in mated connection and can be separated only with some force being applied.

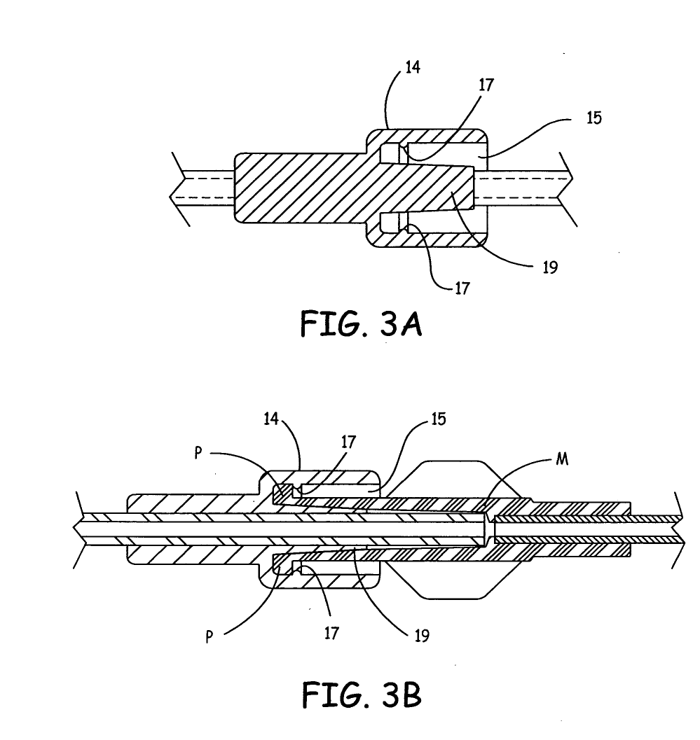

FIG. 3A depicts a cutaway side view of a first connection element 14, according to an alternative embodiment of the present invention. FIG. 3B depicts a cutaway side view of the first connection element 14 of FIG. 3A in mated connection with a male portion M of a catheter, according to one embodiment of the present invention. The connection element 14, according to one embodiment, is a “female” connection element having an opening 15 at one end configured to receive an appropriate “male” connection portion of a catheter. Inside the opening 15, the connection element 14 has two small protruding elements 17 and a sealing element 19. The protruding elements 17 are configured to contact protruding elements P on the male element M such that the male element M and the first connection element 14 are held in mated connection and can be separated only with some force being applied. The sealing element 19 is configured to provide a tighter mating connection or “seal” between the first connection element 14 and the male element M by contacting and maintaining contact with an inner portion of the male element M.

Returning to FIG. 1, the second connection element 16, according to one embodiment, is associated with the elongated tubular member 12 at the proximal end of the tubular member 12. The second connection element 16, according to one embodiment, is a lure connector. Alternatively, the second connection element is any known connection element. The second connection element 16 may be used to evacuate air and flush the loader with fluids. Alternatively, the second connection element 16 is used to attach tools such as compression devices or catheters. In a further alternative, the second connection element 16 is used both to evacuate air and flush fluids and to attach tools.

The second connection element 16 can vary in size. According to one embodiment, the second connection element accepts devices of sizes varying from about size 3 French to about size 16 French. Alternatively, the second connection element accepts devices of sizes varying from about size 5 French to about size 12 French. In a further alternative, the second connection element 16 accepts adapters that are configured to accept devices of French sizes that are not compatible with the second connection element 16 itself.

According to one embodiment, the apparatus 10 is any fluoropolymer. For example, according to one embodiment the apparatus 10 is comprised of PTFE. Alternatively, the apparatus 10 is comprised of MFA. Alternatively, the apparatus is a co-extruded material having an inner liner 18 where the inner liner 18 is any fluoropolymer. In a further alternative, the inner liner 18 is any similar low-friction material.

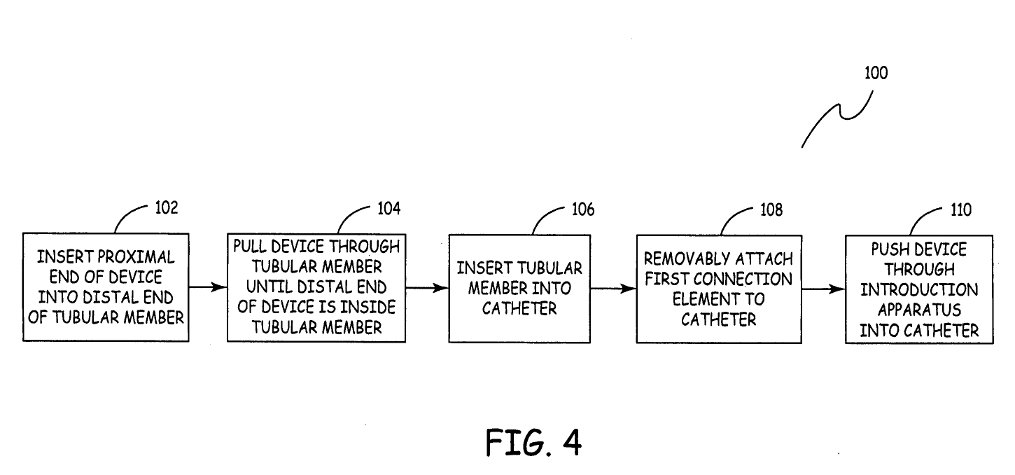

FIG. 4 depicts a method of using an introduction apparatus 100 according to one embodiment of the present invention. In operation, the proximal end of a device is inserted into the distal end of the tubular member 12 (block 102). Once the device has been pulled through the tubular member 12 such that the distal end of the device is enclosed within the tubular member 12 (block 104), the tubular member 12 is inserted into a catheter (block 106). Upon insertion, the first connection element 14 removably attaches to the catheter (block 108). At this point, the device is pushed through the introduction apparatus 10 and into the catheter to which the apparatus 10 is attached (block 110).

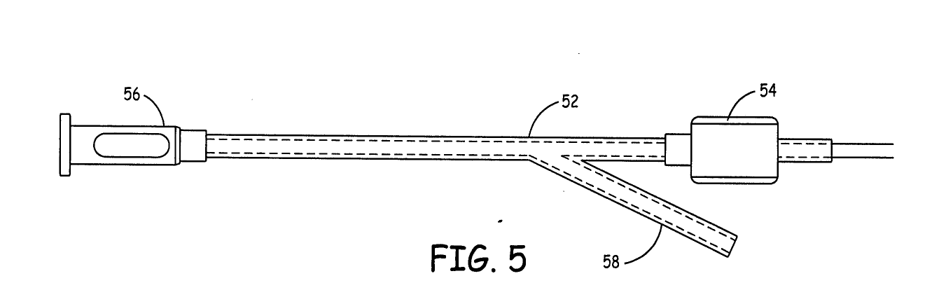

FIG. 5 depicts an introduction apparatus 50 according to an alternative embodiment of the present invention. The apparatus 50 has an elongated tubular member 52 and a first connection element 54 and a second connection element 56. The apparatus also has an additional tubular arm 58 associated with the tubular member. According to one embodiment, the tubular arm 58 is configured to accept a device for insertion into a catheter while the apparatus 50 is already attached to the catheter. In a further embodiment, the introduction apparatus 50 has an elongated tubular member 52, a tubular arm 58, and a second connection element 56 and is permanently attached at the distal end of the tubular member 52 to the catheter.

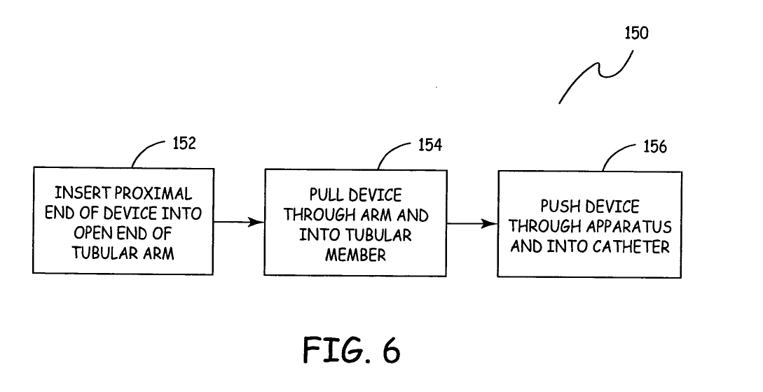

FIG. 6 depicts a method of using an introduction apparatus (50) 150 with a tubular arm 58 according to one embodiment of the present invention where the apparatus 50 is already attached to the catheter. In operation, the proximal end of a device is inserted into the open end of the tubular arm 58 (block 152). Once the device has been pulled through the tubular arm 58 and into the tubular member 52 such that the distal end of the device is enclosed within the tubular member 12 (block 154), the device is pushed through the introduction apparatus 10 and into the catheter to which the apparatus 10 is attached (block 156).

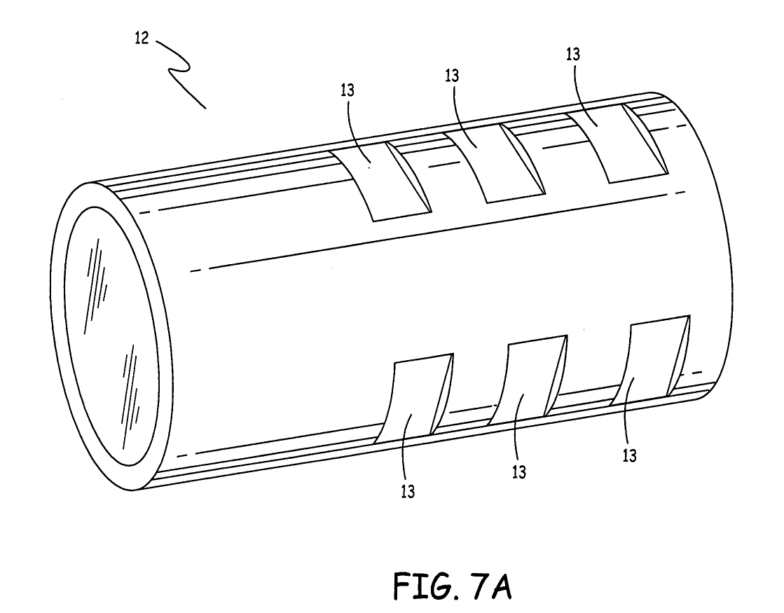

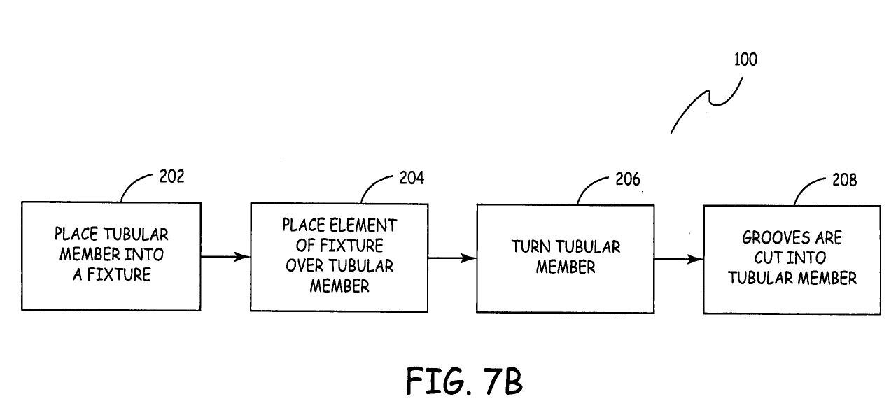

The tubular member 12 according to one embodiment is made by a known extrusion process. FIG. 7A depicts a portion of a tubular member 12 made by a process that results in a configuration that allows adherence of external elements, according to an alternative embodiment of the present invention. That is, a grooving process, which is also known as a “roughing” process, is used to create grooves 13 in the tubular member 12 which are configured to allow for the attachment of external elements, such as the first connection element 14 or the second connection element 16.

FIG. 7B depicts a grooving process 200 used to make grooves in the tubular member 12, according to one embodiment of the present invention. The tubular member 12 is placed into a fixture (block 202). Upon placement in the fixture, an element of the fixture is placed over the tubular member 12 such that grinding wheels contact the tubular member 12 at desired locations (block 204). Next, the tubular member is turned (block 206), thus causing grooves to be cut into the tubular member (block 208).

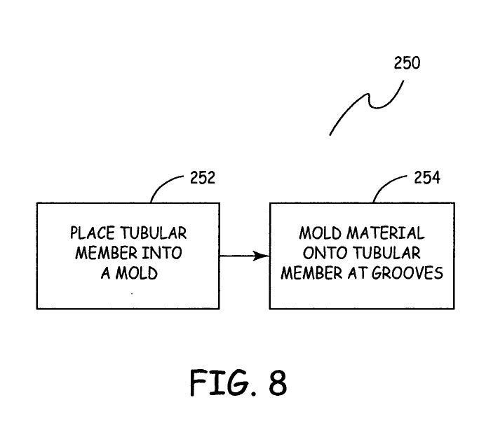

FIG. 8 depicts a method of adding external elements to the tubular member (12) 250, according to one embodiment of the invention. For example, the first connection element 14 and the second connection element 16 can be added by this process. The tubular member 12, having grooves at appropriate locations, is placed into a mold (block 252). Then an appropriate material is molded onto the tubular member 12 at the grooves (block 254).

In a further alternative, if the apparatus 10 was created using a co-extrusion process, the tubular member 12 may not need grooving if the outer layer 20 is configured to allow adherence of external elements.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

We claim:

1. A medical device introduction apparatus comprising:
a co-extruded elongated tubular member, the elongated tubular member having an inner liner having PTFE;
a first connection element associated with a distal portion of the elongated tubular member, the first connection element configured to be removably attachable to a catheter; and
a second connection element associated with a proximal end of the elongated tubular member, the second connection element configured to receive a medical device.

2. The device of claim 1 wherein the first connection element is near a distal end of the elongated tubular member.

3. The device of claim 1 wherein the first connection element is screw connector.

4. The device of claim 1 wherein the first connection element is a snap connector.

5. The device of claim 1 wherein the elongated tubular member is PTFE.

6. The device of claim 1 further comprising a tubular arm associated with the elongated tubular member.

7. A method of introducing a medical device into a catheter comprising:
inserting a distal end of an elongated tubular member into a catheter;
removably attaching a first connection element to the catheter upon inserting the distal end a predetermined distance into the catheter; and
inserting the medical device into a second connection element at a proximal end of the elongated tubular member.
